(19) United States
(12) Patent Application Publication
(10) Pub. No.: US 2024/0139200 A1
(43) Pub. Date: May 2, 2024

(54) COMBINATION OF REBOXETINE AND A MUSCARINIC RECEPTOR ANTAGONIST (MRA) FOR USE IN TREATING SLEEP APNEA

(71) Applicant: APNIMED, INC. (DELAWARE), Cambridge, MA (US)

(72) Inventors: Lawrence G. MILLER, Cambridge, MA (US); Ronald FARKAS, Clarksiville, MD (US); Luigi TARANTO-MONTEMURRO, Brookline, MA (US)

(21) Appl. No.: 18/548,604

(22) PCT Filed: Mar. 3, 2022
(86) PCT No.: PCT/US2022/018604

§ 371 Date: Sep. 1, 2023

Related U.S. Application Data
(60) Provisional application No. 63/156,463, filed on Mar. 4, 2021.

(51) Int. Cl. A61K 31/5375; A61K 31/216; A61P 11/16

(52) U.S. Cl. CPC A61K 31/5375; A61K 31/216; A61P 11/16

(57) ABSTRACT

Pharmaceutical compositions comprising reboxetine or a pharmaceutically acceptable salt thereof and a muscarinic receptor antagonist (MRA) and methods of treating sleep apnea comprising administering reboxetine or a pharmaceutically acceptable salt thereof and an MRA are described herein. In some embodiments, the MRA is oxybutynin or (R)-oxybutynin or a pharmaceutically acceptable salt thereof.

### CROSS-REFERENCE TO RELATED APPLICATION

This application claims priority to U.S. provisional application 63/156,463, filed Mar. 4, 2021, the entire contents of which are incorporated herein by reference.

### TECHNICAL FIELD

The present invention provides pharmaceutical compositions comprising reboxetine or a pharmaceutically acceptable salt thereof and a muscarinic receptor antagonist (MRA) and methods of treating sleep apnea comprising administering reboxetine or a pharmaceutically acceptable salt thereof and an MRA.

### BACKGROUND

Obstructive Sleep Apnea (OSA) is a common disorder caused by collapse of the pharyngeal airway during sleep. OSA can have serious health consequences.

### SUMMARY

One aspect of the present invention provides a method of treating a subject having a condition associated with pharyngeal airway collapse, the method comprising administering to a subject in need thereof an effective amount of (i) reboxetine or a pharmaceutically acceptable salt thereof and (ii) a muscarinic receptor antagonist (MRA).

Embodiments of this aspect of the invention may include one or more of the following optional features. In some embodiments, the reboxetine or pharmaceutically acceptable salt thereof is administered at a dosage of from about 1 mg to about 8 mg. In some embodiments, the reboxetine or pharmaceutically acceptable salt thereof is administered at a dosage of from about 2 mg to about 6 mg. In some embodiments, the MRA and reboxetine are each administered daily. In some embodiments, the MRA and reboxetine are administered in a single composition. In some embodiments, single composition is an oral administration form. In some embodiments, the oral administration form is a syrup, pill, tablet, troche, capsule, or patch. In some embodiments, the MRA is selected from the group consisting of atropine, propantheline, bethanechol, solifenacin, darifenacin, tolterodine, fesoterodine, trospium, and oxybutynin, or a pharmaceutically acceptable salt thereof. In some embodiments, the MRA is selected from the group consisting of anisotropine, benztropine, biperiden, clidinium, cycrimine, dicyclomine, diphemanil, diphenidol, ethopropazine, glycopyrrolate, hexocyclium, isopropamide, mepenzolate, methixene, methscopolamine, oxyphencyclimine, oxyphenonium, procyclidine, scopolamine, tridihexethyl, and trihexyphenidyl, or a pharmaceutically acceptable salt thereof. In some embodiments, the MRA is oxybutynin or a pharmaceutically acceptable salt thereof. In some embodiments, the oxybutynin or pharmaceutically acceptable salt thereof is administered at a dosage of from about 1 mg to about 25 mg. In some embodiments, the oxybutynin or pharmaceutically acceptable salt thereof is administered at a dosage of from about 2 mg to about 15 mg. In some embodiments, the MRA is (R)-oxybutynin or a pharmaceutically acceptable salt thereof. In some embodiments, the (R)-oxybutynin or pharmaceutically acceptable salt thereof is administered at a dosage of from about 1 mg to about 25 mg. In some embodiments, the (R)-oxybutynin or pharmaceutically acceptable salt thereof is administered at a dosage of from about 2 mg to about 15 mg. In some embodiments, the MRA is fesoterodine. In some embodiments, the reboxetine or pharmaceutically acceptable salt thereof is (S,S)-reboxetine or a pharmaceutically acceptable salt thereof. In some embodiments, the MRA is administered daily. In some embodiments, the condition associated with pharyngeal airway collapse is sleep apnea, e.g., obstructive sleep apnea (OSA). In some embodiments, the condition associated with pharyngeal airway collapse is snoring, e.g., simple snoring. In some embodiments, the subject is in a non-fully conscious state. In some embodiments, the non-fully conscious state is sleep.

Another aspect of the invention provides a pharmaceutical composition comprising (i) reboxetine or a pharmaceutically acceptable salt thereof and (ii) a muscarinic receptor antagonist (MRA), and (iii) a pharmaceutically acceptable carrier.

Embodiments of this aspect of the invention may include one or more of the following optional features. In some embodiments, the reboxetine or pharmaceutically acceptable salt thereof is present in an amount of from about 1 mg to about 8 mg. In some embodiments, the reboxetine or pharmaceutically acceptable salt thereof is present in an amount of from about 2 mg to about 6 mg. In some embodiments, the MRA is selected from the group consisting of atropine, propantheline, bethanechol, solifenacin, darifenacin, tolterodine, fesoterodine, trospium, and oxybutynin, or or a pharmaceutically acceptable salt thereof. In some embodiments, the MRA is selected from the group consisting of anisotropine, benztropine, biperiden, clidinium, cycrimine, dicyclomine, diphemanil, diphenidol, ethopropazine, glycopyrrolate, hexocyclium, isopropamide, mepenzolate, methixene, methscopolamine, oxyphencyclimine, oxyphenonium, procyclidine, scopolamine, tridihexethyl, and trihexyphenidyl, or a pharmaceutically acceptable salt thereof. In some embodiments, the MRA is oxybutynin or a pharmaceutically acceptable salt thereof. In some embodiments, the oxybutynin or pharmaceutically acceptable salt thereof is present in amount of from about 1 mg to about 25 mg. In some embodiments, the oxybutynin or pharmaceutically acceptable salt thereof is present in amount of from about 2 mg to about 15 mg. In some embodiments, the MRA is (R)-oxybutynin or a pharmaceutically acceptable salt thereof. In some embodiments, the (R)-oxybutynin or pharmaceutically acceptable salt thereof is present in amount of from about 1 mg to about 25 mg. In some embodiments, the (R)-oxybutynin or pharmaceutically acceptable salt thereof is present in amount of from about 2 mg to about 15 mg. In some embodiments, the MRA is fesoterodine. In some embodiments, the reboxetine or pharmaceutically acceptable salt thereof is (S,S)-reboxetine or a pharmaceutically acceptable salt thereof. In some embodiments, the composition is an oral administration form. In some embodiments, the oral administration form is a syrup, pill, tablet, troche, capsule, or patch. In some embodiments, the composition is for use in treating a subject having a condition associated with pharyngeal airway collapse. In some embodiments, the condition associated with pharyngeal airway collapse is sleep apnea, e.g., obstructive sleep apnea (OSA). In some embodiments, the condition associated with pharyngeal airway collapse is snoring, e.g., simple snoring. In some embodiments, the subject is in a non-fully conscious state. In some embodiments, the non-fully conscious state is sleep.

Another aspect of the invention provides reboxetine or a pharmaceutically acceptable salt thereof and a muscarinic receptor antagonist (MRA) for use in treating a subject having a condition associated with pharyngeal airway collapse.

Another aspect of the invention provides a kit comprising reboxetine or a pharmaceutically acceptable salt thereof and a muscarinic receptor antagonist (MRA). In some embodiments, the kit is for use in treating a subject having a condition associated with pharyngeal airway collapse.

Another aspect of the invention provides a therapeutic combination of reboxetine or a pharmaceutically acceptable salt thereof and a muscarinic receptor antagonist (MRA) for use in treating a subject having a condition associated with pharyngeal airway collapse.

Unless otherwise defined, all technical and scientific terms used herein have the same meaning as commonly understood by one of ordinary skill in the art to which this invention belongs. Methods and materials are described herein for use in the present invention; other suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

The following figures are provided by way of example and are not intended to limit the scope of the claimed invention.

FIG. 1 is a graphic illustration of an obstructive apnea. The top channel shows the electroencephalogram (EEG) pattern of sleep. The next channel represents airflow. The next three channels show ventilator effort by movements of the rib cage and abdomen and changes in esophageal pressure, all of which reflect a respiratory effort against an occluded upper airway. The last channel indicates oxyhemoglobin saturation.

FIG. 2 is a plot showing the total apnea-hypopnea index (AHI) of the individual subjects after placebo and after reb-oxy treatment for the study of Example 1.

FIG. 3 is a bar chart showing the AHI reduction from baseline for subjects in the placebo and treatment (reb4-oxy5) groups for the study of Example 1.

FIG. 4 is a plot showing the hypoxic burden (HB) of the individual subjects after placebo and after reb-oxy treatment for the study of Example 1.

FIG. 5 is a bar chart showing the HB reduction from baseline for subjects in the placebo and treatment (reb4-oxy5) groups for the study of Example 1.

### DETAILED DESCRIPTION

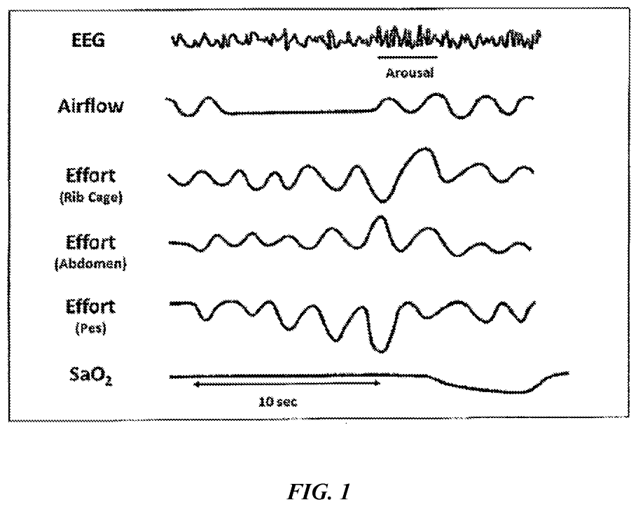

In humans, the pharyngeal airway region has no bone or cartilage support, and it is held open by muscles. When these muscles relax during sleep, the pharynx can collapse resulting in cessation of airflow. As shown in FIG. 1, ventilatory effort continues and increases in an attempt to overcome the obstruction, shown by an increase in esophageal pressure change. Rib cage and abdominal movements are in the opposite direction as a result of the diaphragm contracting against an occluded airway, forcing the abdominal wall to distend out and the chest wall to cave inward.

Increasing efforts to breathe lead to an arousal from sleep, visualisable on an EEG (FIG. 1), and result in opening of the airway and a resumption of normal breathing. The lack of airflow during the apnea also causes hypoxia, shown by a drop in oxyhemoglobin saturation (FIG. 1). Severity is generally measured using the apnea-hypopnea index (AHI), which is the combined average number of apneas (cessation of breathing for at least ten seconds) and hypopneas (reduced airflow and oxygen saturation) that occur per hour of sleep (Ruehland et al., The new AASM criteria for scoring hypopneas: Impact on the apnea hypopnea index. SLEEP 2009; 32(2):150-157).

FIG. 1 is a graphic illustration of an obstructive apnea. The top channel shows the electroencephalogram (EEG) pattern of sleep. The next channel represents airflow. The next three channels show ventilatory effort by movements of the rib cage and abdomen and changes in esophageal pressure, all of which reflect a respiratory effort against an occluded upper airway. The last channel indicates oxyhemoglobin saturation.

When a stringent definition of OSA is used (an AHI of >15 events per hour or AHI >5 events per hour with daytime sleepiness), the estimated prevalence is approximately 15 percent in males and 5 percent in females. An estimated 30 million individuals in the United States have OSA, of which approximately 6 million have been diagnosed. The prevalence of OSA in the United States appears to be increasing due to aging and increasing rates of obesity. OSA is associated with major comorbidities and economic costs, including: hypertension, diabetes, cardiovascular disease, motor vehicle accidents, workplace accidents, and fatigue/lost productivity. (Young et al., WMJ 2009; 108:246; Peppard et al., Am J Epidemiol 2013; 177:1006.)

The present leading treatment is continuous positive airway pressure (CPAP). CPAP is effective in virtually all patients, and approximately 85% of diagnosed patients are prescribed CPAP, but compliance is low. Patients find CPAP uncomfortable and often intolerable; at least 30% of patients (up to 80%) are regularly non-adherent and thus untreated (Weaver, Proc Am Thorac Soc. 2008 Feb. 15; 5(2): 173-178). Other treatment modalities with variable rates of success include oral appliances (10%) and surgery (5%), but neither is likely to be effective across the general population.

The search for medicines to activate pharyngeal muscles in sleeping humans has been discouraging; agents such as serotonin reuptake inhibitors, tricyclic antidepressants, and sedatives have all been tested in humans and shown to be ineffective at reducing OSA severity. See, e.g., Proia and Hudgel, Chest. 1991 August; 100(2):416-21; Brownell et al., N Engl J Med 1982, 307:1037-1042; Sangal et al., Sleep Med. 2008 July; 9(5):506-10. Epub 2007 Sep. 27; Marshall et al. p. 2008 June; 31(6):824-31; Eckert et al., Clin Sci (Lond). 2011 June; 120(12); 505-14; Taranto-Montemurro et al., Sleep. 2017 Feb. 1; 40(2).

Methods of Treatment

The methods described herein include methods for the treatment of disorders associated with pharyngeal airway muscle collapse during sleep. In some embodiments, the disorder is obstructive sleep apnea (OSA) or simple snoring. Generally, the methods include administering a therapeutically effective amount of reboxetine or a pharmaceutically acceptable salt thereof and a muscarinic receptor antagonist (MRA) as known in the art and/or described herein, to a subject who is in need of, or who has been determined to be in need of, such treatment.

As used in this context, to “treat” means to ameliorate at least one symptom of the disorder associated with pharyngeal airway collapse. Often, pharyngeal airway collapse during sleep results in snoring and/or an interruption in breathing (apnea or hypopnea), arousal from sleep, and reduced oxygenation (hypoxemia); thus, a treatment can result in a reduction in snoring, apneas/hypopneas, sleep fragmentation, and hypoxemia. Administration of a therapeutically effective amount of a compound described herein for the treatment of a subject with OSA may result in decreased AHI. Measurement of OSA disease and symptoms may be, for example, by polysomnography (PSG).

In general, an “effective amount” of a compound refers to an amount sufficient to elicit the desired biological response, e.g., to treat a condition associated with pharyngeal airway collapse, e.g., to treat sleep apnea or snoring. As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject. An effective amount encompasses therapeutic and prophylactic treatment.

An effective amount can be administered in one or more administrations, applications or dosages. The compositions can be administered from one or more times per day to one or more times per week; including once every other day. In some embodiments, the compositions are administered daily. In some embodiments, the compositions are administered daily before sleep time, e.g., immediately before sleep time or 15-60 minutes before sleep time. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, the terms “subject” and “patient” are used interchangeably. The terms “subject” and “patient” refer to an animal (e.g., a bird such as a chicken, quail or turkey, or a mammal), specifically a “mammal” including a non-primate (e.g., a cow, pig, horse, sheep, rabbit, guinea pig, rat, cat, dog, and mouse) and a primate (e.g., a monkey, chimpanzee and a human), and more specifically a human. In one embodiment, the subject is a non-human animal such as a farm animal (e.g., a horse, cow, pig or sheep), or a pet (e.g., a dog, cat, guinea pig or rabbit). In a preferred embodiment, the subject is a human.

As used herein, “pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

As used herein, the term “unit dosage form” is defined to refer to the form in which the compound is administered to a subject. Specifically, the unit dosage form can be, for example, a pill, capsule, or tablet. In some embodiments, the unit dosage form is a capsule.

As used herein, “solid dosage form” means a pharmaceutical dose(s) in solid form, e.g. tablets, capsules, granules, powders, sachets, reconstitutable powders, dry powder inhalers and chewables.

For the compounds disclosed herein, single stereochemical isomers, as well as enantiomers, diastereomers, cis/trans conformation isomers, and rotational isomers, and racemic and non-racemic mixtures thereof, are within the scope of the invention. Unless otherwise indicated, all tautomeric forms of the compounds disclosed herein are within the scope of the invention.

Reboxetine is the generic name of the pharmaceutical substance with the chemical name of 2-((2-ethoxyphenoxy)(phenyl)methyl)morpholine or 2-[α-(2-ethoxyphenoxy)benzyl]-morpholine, and its pharmaceutically acceptable salts. In various embodiments, reboxetine may be a racemic mixture of R,R- and S,S-enantiomers, or an isolated enantiomer, e.g., the S,S-enantiomer. In some embodiments, reboxetine may be reboxetine hydrochloride.

In some embodiments, the methods include administering a dose of from about 0.2 mg to about 12 mg of reboxetine or a pharmaceutically acceptable salt thereof. In some embodiments, the dose of reboxetine or a pharmaceutically acceptable salt thereof is from about 1 mg to about 8 mg. In some embodiments, the dose of reboxetine or pharmaceutically acceptable salt thereof is from about 0.5 mg to about 6 mg. In some embodiments, the dose of reboxetine or pharmaceutically acceptable salt thereof is from about 2 mg to about 6 mg. In some embodiments, the dose of reboxetine or pharmaceutically acceptable salt thereof is about 4 mg. In some embodiments, the dose of reboxetine or pharmaceutically acceptable salt thereof is about 6 mg. In some embodiments, the dose of reboxetine or pharmaceutically acceptable salt thereof is about 2 mg. In some embodiments, the dose of reboxetine or pharmaceutically acceptable salt thereof is about 3 mg. In some embodiments, the reboxetine or pharmaceutically acceptable salt thereof is (S,S)-reboxetine or a pharmaceutically acceptable salt thereof.

In methods comprising administration of oxybutynin or (R)-oxybutynin or a pharmaceutically acceptable salt thereof (or another MRA), the dose of oxybutynin or (R)-oxybutynin or pharmaceutically acceptable salt thereof may be from about 1 mg to about 25 mg (or a dose equivalent thereof of another MRA), or in some embodiments, from about 2 mg to about 15 mg. In some embodiments, the dose of oxybutynin or pharmaceutically acceptable salt thereof is from about 2.5 mg to about 10 mg, e.g., 5 mg. In some embodiments, the dose of (R)-oxybutynin or pharmaceutically acceptable salt thereof is from about 1 mg to about 10 mg, e.g., 2.5 mg. In some embodiments, the dose of oxybutynin or (R)-oxybutynin or pharmaceutically acceptable salt thereof is from about 1 mg to about 5 mg.

In some embodiments, the methods include administering 4 mg reboxetine hydrochloride and 5 mg oxybutynin chloride. In some embodiments, the methods include administering 4 mg reboxetine hydrochloride and 5 mg (R)-oxybutynin chloride. In some embodiments, the methods include administering 4 mg reboxetine hydrochloride and 2.5 mg (R)-oxybutynin chloride. In some embodiments, the methods include administering 6 mg reboxetine hydrochloride and 5 mg oxybutynin chloride. In some embodiments, the methods include administering 6 mg reboxetine hydrochloride and 5 mg (R)-oxybutynin chloride. In some embodiments, the methods include administering 6 mg reboxetine hydrochloride and 2.5 mg (R)-oxybutynin chloride.

Pharmaceutical Compositions

Also provided herein are pharmaceutical compositions comprising reboxetine or a pharmaceutically acceptable salt thereof and an MRA as active ingredients. The MRA and reboxetine can be in a single composition or in separate compositions.

Exemplary muscarinic receptor antagonists (MRAs) include atropine, propantheline, bethanechol, solifenacin, darifenacin, tolterodine, fesoterodine, trospium, and oxybutynin, or pharmaceutically acceptable salts thereof, which have activity on the M2 receptor. Other exemplary antimuscarinics include anisotropine, benztropine, biperiden, clidinium, cycrimine, dicyclomine, diphemanil, diphenidol, ethopropazine, glycopyrrolate, hexocyclium, isopropamide, mepenzolate, methixene, methscopolamine, oxyphencyclimine, oxyphenonium, procyclidine, scopolamine, tridihexethyl, and trihexyphenidyl, or pharmaceutically acceptable salts thereof.

In some embodiments, the muscarinic receptor antagonist is oxybutynin or (R)-oxybutynin, or a pharmaceutically acceptable salt thereof. As used herein, (R)-oxybutynin refers to the (R)-oxybutynin stereoisomer substantially free of other stereoisomers of oxybutynin. In some embodiments, the muscarinic receptor antagonist is fesoterodine.

In some embodiments, the reboxetine or pharmaceutically acceptable salt thereof is (S,S)-reboxetine or a pharmaceutically acceptable salt thereof. As used herein, (S,S)-reboxetine refers to the (S,S)-reboxetine stereoisomer substantially free of other stereoisomers of reboxetine.

Pharmaceutical compositions typically include a pharmaceutically acceptable carrier. As used herein the language “pharmaceutically acceptable carrier” includes saline, solvents, dispersion media, diluents, fillers, coatings, antibacterial and antifungal agents, isotonic and absorption delaying agents, and the like, compatible with pharmaceutical administration.

The active pharmaceutical ingredients (APIs) for use in the present invention may be provided as pharmaceutically acceptable salts. For example, in some embodiments, oxybutynin is oxybutynin chloride. In some embodiments, (R)-oxybutynin is (R)-oxybutynin chloride. In some embodiments, reboxetine is reboxetine hydrochloride.

Oxybutynin is the generic name for the pharmaceutical substance with the chemical name 4-diethylamino-2-butynylphenylcyclohexylglycolate or 4-(diethylamino)but-2-ynyl 2-cyclohexyl-2-hydroxy-2-phenylacetate, and its pharmaceutically acceptable salts. In various embodiments, oxybutynin may be a racemic mixture of R- and S-enantiomers, or an isolated enantiomer, e.g., the R-enantiomer. In various embodiments, oxybutynin may be oxybutynin chloride or (R)-oxybutynin chloride.

Pharmaceutical compositions are typically formulated to be compatible with its intended route of administration. Examples of routes of administration include systemic oral or transdermal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, oral compositions generally include an inert diluent or an edible carrier. For the purpose of oral therapeutic administration, the active compound(s) can be incorporated with excipients and used in the form of pills, tablets, troches, or capsules, e.g., gelatin capsules. Oral compositions can also be prepared using a fluid carrier. In some embodiments, a composition according to the present invention may be a unit dosage form. In some embodiments, a composition according to the present invention may be a solid dosage form, e.g., a tablet or capsule.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration of the compounds as described herein can also be by transdermal means, e.g., using a patch, gel, or lotion, to be applied to the skin. For transdermal administration, penetrants appropriate to the permeation of the epidermal barrier can be used in the formulation. Such penetrants are generally known in the art. For example, for transdermal administration, the active compounds can formulated into ointments, salves, gels, or creams as generally known in the art. The gel and/or lotion can be provided in individual sachets, or via a metered-dose pump that is applied daily; see, e.g., Cohn et al., Ther Adv Urol. 2016 April; 8(2): 83-90.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration or use in a method described herein.

### EXAMPLES

The invention is further described in the following examples, which do not limit the scope of the invention described in the claims.

### Example 1. A Randomized, Placebo-Controlled, Double-Blind Crossover, 1-Week Trial

Methods: A randomized, placebo-controlled, double-blind, crossover trial was performed, comparing 4 mg reboxetine plus 5 mg oxybutynin (reb-oxy) to placebo administered orally before sleep in OSA subjects. Patients with a previous diagnosis of moderate-to-severe OSA performed a baseline in-lab polysomnography (PSG) to compare AHI and sleep characteristics after 7 nights of placebo and 7 nights of reb-oxy. A psychomotor vigilance test (PVT) and questionnaires on sleepiness and quality of life were also administered after 7 days of treatment. A home-oxymeter was provided for the entire at-home period to evaluate the frequency of oxygen desaturations (ODI) ≥4%. Reb-oxy effect on specific OSA pathophysiology traits was also analyzed.

The study design was as follows: up to 28 days to conduct screening and baseline PSG, followed by 7 days randomized at-home study treatment, then an in-lab PSG night, followed by 7 days washout (up to 10 days if necessary, for scheduling), then 7 days crossover to the other treatment arm, and a final in-lab PSG night.

Two blinded capsules were taken each night of study treatment. On nights of study treatment, 1 capsule of reboxetine and 1 capsule of oxybutynin, or of corresponding placebo, were taken at the participant's bedtime (QHS).

| Study Treatment | Reboxetine | |
|---|---|---|
| Name: | hydrochloride | Oxybutynin chloride |
| Dosage Formulation: | capsule | capsule |
| Dosage Level: | 4 mg | 5 mg |
| Route of Administration: | Oral | Oral |
| Dosing Instructions: | 1 capsule QHS with at | 1 capsule QHS with at |
| | least 20 mL water | least 20 mL water |

Results: 16 subjects with (median [interquartile range]) age 57 [51-61] years and body mass index 30 [26-36] kg/m² completed the study. AHI went from 49 [35-57] events/h at baseline to 18 [13-21] events/h (59% median reduction) on reb-oxy and to 39 [29-48] events/h (6% median reduction) on placebo (p<0.001). On reb-oxy 81% of subjects reduced AHI by more than 50% compared to 13% on placebo. 37% of subjects presented an AHI less than 15/h compared to 6% on placebo. PVT decreased from 250 [239-312] msec on baseline to 223 [172-244] on reb-oxy versus 264 [217-284] on placebo (p<0.001). Subjective quality of life and sleepiness were not different between treatment groups. Both at-home ODI and in-lab ODI improved on reb-oxy versus placebo (p<0.001 and p=0.021, respectively). Analysis of pathophysiology traits showed that reb-oxy increased muscle compensation and reduced arousal threshold compared to placebo (p=0.012 and p=0.01, respectively).

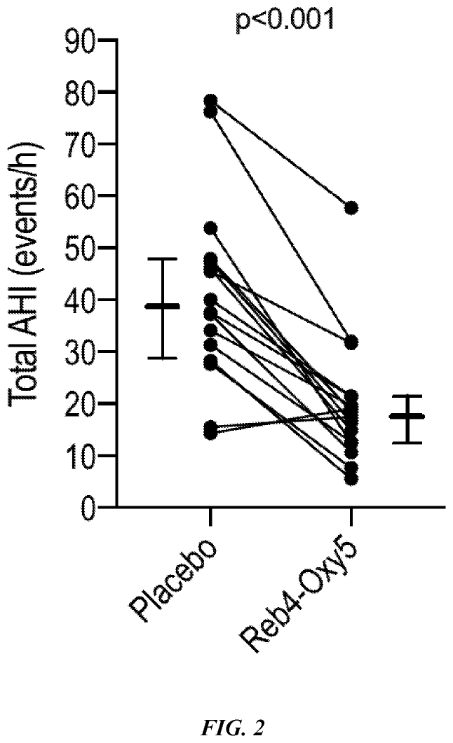

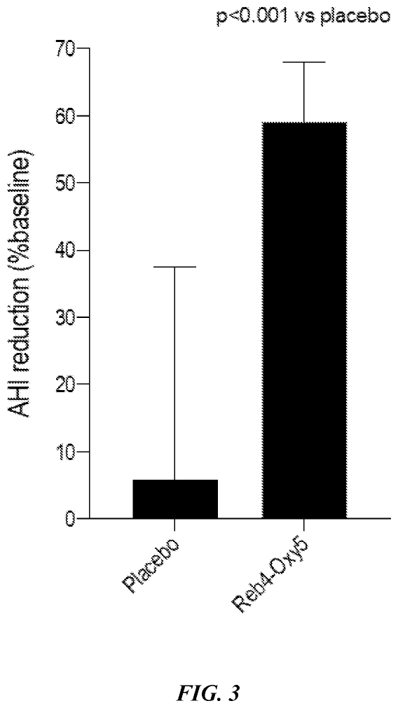

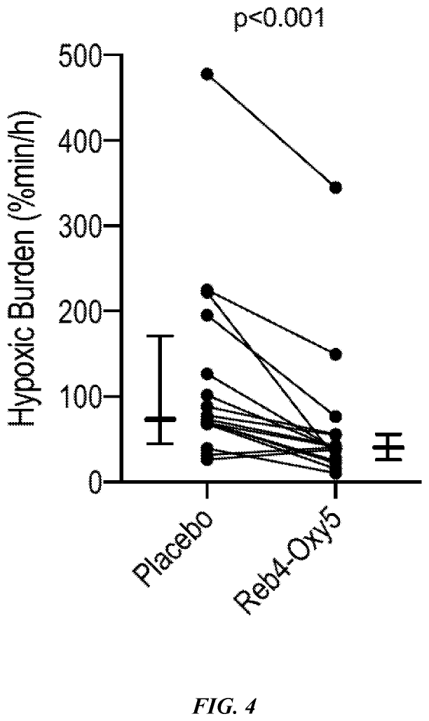

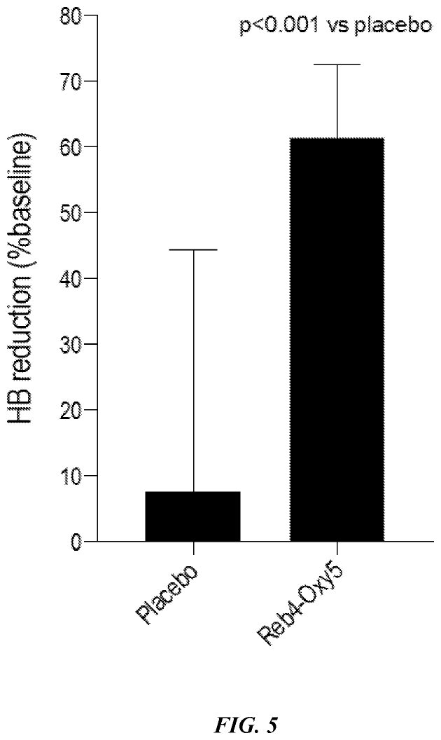

FIG. 2 shows the total AHI of the individual subjects after placebo and after reb-oxy treatment. FIG. 3 shows the improvement in AHI reduction from baseline for reb-oxy compared to placebo. Hypoxic burden (HB) was also measured and calculated during the study. Hypoxic burden was measured and calculated as described in A. Azarbarzin et al., The hypoxic burden of sleep apnoea predicts cardiovascular disease-related mortality: the Osteoporotic Fractures in Men Study and the Sleep Heart Health Study, Eur Heart J., 2019 Apr. 7; 40(14): 1149-1157. FIG. 4 shows the hypoxic burden of the individual subjects after placebo and after reb-oxy treatment. FIG. 5 shows the improvement in HB reduction from baseline for reb-oxy compared to placebo.

Conclusions: The administration of reboxetine plus oxybutynin before bedtime greatly decreased OSA severity and increased objective vigilance over one week of therapy.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a subject having a condition associated with pharyngeal airway collapse, the method comprising administering to a subject in need thereof an effective amount of (i) reboxetine or a pharmaceutically acceptable salt thereof and (ii) a muscarinic receptor antagonist (MRA).

2. The method of claim 1, wherein the reboxetine or pharmaceutically acceptable salt thereof is administered at a dosage of from about 1 mg to about 8 mg.

3. The method of claim 1, wherein the reboxetine or pharmaceutically acceptable salt thereof is administered at a dosage of from about 2 mg to about 6 mg.

4. The method of claim 1, wherein the MRA and reboxetine or pharmaceutically acceptable salt thereof are each administered daily.

5. The method of claim 1, wherein the MRA and reboxetine or pharmaceutically acceptable salt thereof are administered in a single composition, wherein the single composition is an oral administration form.

6. (canceled)

7. (canceled)

8. The method of claim 1, wherein the MRA is selected from the group consisting of atropine, propantheline, bethanechol, solifenacin, darifenacin, tolterodine, fesoterodine, trospium, and oxybutynin, or a pharmaceutically acceptable salt thereof.

9. (canceled)

10. The method of claim 8, wherein the MRA is oxybutynin or a pharmaceutically acceptable salt thereof.

11. The method of claim 10, wherein the MRA is (R)-oxybutynin or a pharmaceutically acceptable salt thereof.

12. The method of claim 10, wherein the oxybutynin or pharmaceutically acceptable salt thereof is administered at a dose of from about 1 mg to about 25 mg.

13. The method of claim 12, wherein the oxybutynin or pharmaceutically acceptable salt thereof is administered at a dose of from about 2 mg to about 15 mg.

14. The method of claim 11, wherein the (R)-oxybutynin or pharmaceutically acceptable salt thereof is administered at a dose of from about 1 mg to about 25 mg.

15. The method of claim 14, wherein the (R)-oxybutynin or pharmaceutically acceptable salt thereof is administered at a dose of from about 2 mg to about 15 mg.

16. The method of claim 1, wherein the reboxetine or pharmaceutically acceptable salt thereof is (S,S)-reboxetine or a pharmaceutically acceptable salt thereof.

17. The method of claim 1, wherein the condition associated with pharyngeal airway collapse is sleep apnea.

18. The method of claim 17, wherein the condition associated with pharyngeal airway collapse is obstructive sleep apnea (OSA).

19. The method of claim 1, wherein the condition associated with pharyngeal airway collapse is snoring.

20. (canceled)

21. The method of claim 1, wherein the subject is in a non-fully conscious state, wherein the non-fully conscious state is sleep.

22. (canceled)

23. A pharmaceutical composition comprising (i) reboxetine or a pharmaceutically acceptable salt thereof and (ii) a muscarinic receptor antagonist (MRA), and (iii) a pharmaceutically acceptable carrier.

24. The composition of claim 23, wherein the reboxetine or pharmaceutically acceptable salt thereof is present in an amount of from about 1 mg to about 8 mg.

25. (canceled)

26. (canceled)

27. (canceled)

28. The composition of claim 23, wherein the MRA is oxybutynin or a pharmaceutically acceptable salt thereof.

29. The composition of claim 28, wherein the MRA is (R)-oxybutynin or a pharmaceutically acceptable salt thereof.

30. The composition of claim 28, wherein the oxybutynin or pharmaceutically acceptable salt thereof is present in amount of from about 1 mg to about 25 mg.

31. (canceled)

32. The composition of claim 29, wherein the (R)-oxybutynin or pharmaceutically acceptable salt thereof is present in amount of from about 1 mg to about 25 mg.

33. (canceled)

34. The composition of claim 23, wherein the reboxetine or pharmaceutically acceptable salt thereof is (S,S)-reboxetine or a pharmaceutically acceptable salt thereof.

35. The composition of claim 23, wherein the composition is an oral administration form.

36. (canceled)

37. (canceled)

38. (canceled)

39. (canceled)

40. (canceled)

41. (canceled)

42. (canceled)

43. (canceled)

44. A kit comprising reboxetine or a pharmaceutically acceptable salt thereof and a muscarinic receptor antagonist (MRA).

45. The kit of claim 44, wherein the MRA is oxybutynin or a pharmaceutically acceptable salt thereof.

46. The kit of claim 45, wherein the MRA is (R)-oxybutynin or a pharmaceutically acceptable salt thereof.

47. (canceled)

48. (canceled)

49. (canceled)

50. (canceled)

51. (canceled)

52. (canceled)

53. (canceled)

54. (canceled)

55. (canceled)
